(19) United States
(12) Patent Application Publication
(10) Pub. No.: US 2011/0237541 A1
(43) Pub. Date: Sep. 29, 2011

(54) TRANSDERMALLY ADMINISTERED ALISKIREN

(76) Inventors: Sylvie Toulmond, Beaconsfield (CA); Elizabeth Kwong, Cranford, NJ (US); Dwight MacDonald, L'lle Bizard (CA)

(21) Appl. No.: 13/131,058

(22) PCT Filed: Nov. 23, 2009
(86) PCT No.: PCT/CA2009/001694

§ 371 Date: May 25, 2011

Related U.S. Application Data
(60) Provisional application No. 61/117,732, filed on Nov. 25, 2008.
(60) Provisional application No. 61/167,186, filed on Apr. 7, 2009.

(51) Int. Cl. A61K 31/165; A61K 31/724; A61P 9/12; A61P 9/10; A61P 9/00; A61N 1/30; A61M 37/00; A61M 5/00; A61M 5/30

(52) U.S. Cl. 514/58; 514/620; 604/20; 604/22; 604/272; 604/68

(57) ABSTRACT

Dosage form of aliskiren, comprising a device for transdermal administration of aliskiren and aliskiren (including salts, prodrugs and metabolites thereof), optionally together with pharmaceutically acceptable carrier(s) to a human being or an animal in order to achieve a desired therapeutic effect. Use of a compound comprising aliskiren, optionally encompassing salts, prodrugs and metabolites thereof, and optionally together with pharmaceutically acceptable carrier(s), for the manufacture of a composition to be administered transdermally for achieving a desired therapeutic effect. Method for achieving a desired therapeutic effect by transdermal administration of a compound comprising aliskiren, optionally encompassing salts, prodrugs and metabolites thereof, and optionally together with pharmaceutically acceptable carrier(s).

### BACKGROUND OF THE INVENTION

Renin released from the kidneys cleaves angiotensinogen to angiotensin I (Ang I), which is then further processed to angiotensin II (Ang II) in the lungs, kidneys and other organs by angiotensin-converting enzyme (ACE). Ang II increases blood pressure; renin inhibitors therefore have an antihypertensive effect due to reductions in the production of Ang I and II. Aliskiren, (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, is a renin-inhibiting compound for treatment of hypertension. The synthesis of aliskiren and its utility for the treatment of hypertension is disclosed in U.S. Pat. No. 5,559,111 (Ciba-Geigy Corporation), which does not disclose transdermal administration of aliskiren. WO 2008023016 discloses a dosage form for transmucosal administration of aliskiren. A 1994 report disclosed transdermal application of the renin inhibitor ciprokiren in squirrel monkeys. (Fischli, et al., HYPERTENSION, 24(2): 163-169 (1994)). Due to low bioavailability afforded by its peptidomimetic nature, oral dosing regimens of aliskiren requires high doses resulting in adverse effects, such as GI disturbances. Because of this, aliskiren is a candidate for transdermal administration, for example, in a patch formulation.

### BRIEF DESCRIPTION OF THE DRAWINGS

FIG. 1 is a comparison of the effect of oral versus transdermal delivery on mean arterial pressure in female double transgenic rats.

### SUMMARY OF THE INVENTION

The present invention relates to transdermal administration of aliskiren, optionally encompassing salts, prodrugs and metabolites thereof for treating hypertension. As such the present invention relates to a transdermal dosage form of aliskiren and a method for treating hypertension of any type, as well as congestive heart failure, angina, myocardial infarction, atherosclerosis, diabetic nephropathy, diabetic cardiac myopathy, renal insufficiency, peripheral vascular disease, left ventricular hypertrophy, cognitive dysfunction and chronic heart failure, comprising the step of administering a therapeutically effective amount of the dosage form for transdermal administration.

Treatment of hypertension in accordance with this invention is achieved through the systemic effect of aliskiren. Aliskiren may be administered in any stereoisomeric form, or mixtures thereof. Transdermal formulations with aliskiren as the active ingredient provide an alternative to the extant formulations for the oral route, and thus overcome some of the difficulties resulting from poor bioavailability in the oral form due to first-pass metabolism or variables such as GI tract pH or gastric emptying. The dermal route also eliminates exposure to the GI membrane, and local GI irritation believed to be caused by aliskiren. Due to more constant plasma/serum concentrations during a dosage interval, peak and trough in blood-drug concentration is minimized, thereby reducing adverse side effects in comparison to tablets/capsules. Clinical efficacy is thus controlled or improved and patient compliance may be thereby enhanced.

The present invention provides a dosage form for transdermal administration of aliskiren, optionally encompassing salts, prodrugs and metabolites thereof, for the treatment of hypertension. This dosage form for transdermal administration, comprises at least one compound for treating hypertension, which is selected from the group consisting of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide and a stereoisomer thereof, or mixture thereof, salts thereof, prodrugs thereof, and metabolites thereof, and a transdermal administration device selected from the group consisting of a reservoir, a matrix, a drug-in-adhesive, a multi-laminate, a polymer-system with no foils, a iontophoretic device, and combinations thereof, an electroporation, a sonophoration, an electroosmosis, an electroincorporation, microneedle and a jet injection device. The present invention further provides a method of treating hypertension by administering aliskiren transdermally. The present invention further provides use of a compound for a desired therapeutic effect, comprising aliskiren for the manufacture of a composition to be administered transdermally for treating hypertension.

### DETAILED DESCRIPTION OF THE INVENTION

The present invention relates to transdermal delivery of renin inhibitors to treat hypertension. Oral administration of peptidomimetic renin inhibitors results in poor bioavailability, which requires use of high doses and higher direct GI exposure. Thus, dosages typically employed result in adverse effects of drug accumulation in the GI tract, including diarrhea.

Transdermal delivery of peptidomimetics such as aliskiren can result in improved bioavailability, reduced GI adverse effects, improved drug potency, sustained & controlled delivery and increased patient compliance. Such delivery can be achieved from topical products such as ointments or creams or from transdermal devices such as reservoir, drug in adhesive matrix, iontophoretic, ultrasonic or microneedle devices.

The present invention relates most particularly to administration via transdermal devices, such as transdermal patches. Devices usable as transdermal patches can be categorized in many different ways, see, e.g., Wick S. Developing A Drug-In-Adhesive Design For Transdermal Drug Delivery. ADHESIVE AGE 1995; 38: 18-24. This reference classifies transdermal devices into four main groups: reservoir type, in which the drug is placed in a liquid or a gel and delivered across a rate-moderating membrane to the skin; matrix type, in which the drug is placed within a non-adhesive polymeric material, typically a hydrogel or soft polymer; drug-in-adhesive type, in which the drug is placed within an adhesive polymer; and multi-laminate type, which is similar to the drug-in-adhesive design but which incorporates an additional layer of pressure sensitive adhesive to cover the entire device and affix it to the skin.

Another type of device, not mentioned by Wick, is the iontophoretic type, which is the predominant mechanism for electrically assisted transdermal delivery. When using the iontophoretic type, an electrical potential gradient is used for transferring the drug through the skin (see, e.g., Singh P et al. Iontophoresis in Drug Delivery: Basic Principles and Applications. CRIT REV THER DRUG CARRIER SYST 1994; 11: 161-213). Additionally, electroporation, electroosmosis, electroincorporation and jet injection can be used. Electroporation is the creation of transient aqueous pores in lipid bilayer membranes by the application of a short electric pulse; skin permeability is thereby altered such that resistance to drug transport is reduced.

Electroporation has been employed in transdermal drug delivery by coupling it with iontophoresis (Bommannan D et al. PHARM RES 1994; 11: 1809-1814, Prausnitz M R et al. PROC NATL ACAD SCI USA 1993; 90: 10504-10508, and Riviere J E et al. J CONTROLLED RELEASE 1995; 36: 299-233). In these cases, a brief pulse of high voltage alters the skin permeability such that subsequent iontophoresis is facilitated. An iontophoretic device suitable for use in the present invention may be manufactured as disclosed in, e.g., Parminder Singh et al, “Iontophoresis in Drug Delivery: Basic Principles and Applications”, CRITICAL REVIEWS IN THERAPEUTIC DRUG CARRIER SYSTEMS, 1994; 11 (2&3):161-213.

With electroosmosis the electric field creates a convective flow of water which allows hydrophilic compounds to be transported. Closely related to electroporation is electroincorporation but here larger particles such microspheres or liposomes are placed on the surface of the skin and subsequent high voltage electrical pulses are employed (Riviere J E and Heit M C. PHARM RES 1997; 14: 687-697). Jet injection can be used both for powders and liquids (Muddle A G et al. PROC LNT SYMP CONTROL. REL. BIOACT. MATER. 1997; 24: 713-714, and Seyam R M et al. UROLOGY 1997, 50: 994-998. By using jet injection, a drug can be administered by a needle-free painless injection.

Ultrasonic delivery, such as taught by, e.g., U.S. Pat. No. 6,842,641, which teaches sonoporation of the skin area for transdermal and/or intradermal delivery of a drug solution is a means contemplated by the present invention as well.

It is important to note that variations and combinations of each type of device are encompassed within the scope of the present invention. E.g., a multi-laminate type device may encompass a device with many layers in a sandwich construction, such as the drug in one layer, excipients in a further layer, a membrane in another layer and an adhesive in still another layer.

Alternatively, the multi-laminate device could be composed of several drug-in-adhesive layers or combinations of the above layers. Any liquid or gel used in a reservoir-type device could be hydrophilic or lipophilic, such as water, alcohols, mineral oils, silicone fluids, various copolymers, such as ethylene vinyl acetate, vinyl acetate or polyvinyl alcohol/polyvinyl pyrrolidone. The reservoir may also include dyes, inert fillers, diluents, antioxidants, anti-irritants, antisensitizers, permeation enhancers, stabilizers, solubilizing agents and other pharmacologically inactive pharmaceutical agents being well known in the art.

Adhesives used are generally rubber, e.g., polyisobutylenes, acrylate and silicone type. The adhesives may be chemically modified, and may have a wide range of molecular weights. Several types of excipients may be added to the adhesives such as fillers, stabilizers, plasticizers, buffering agents, permeation enhancers, permeation retardants, anti-irritants, anti-sensitizers, solubilizing agents and other pharmaceutical ingredients being well known in the art.

Polymer films that may be used for making the rate-moderating membrane 15 include, without limitation, those comprising low- and high-density polyethylene, ethyl vinyl acetate copolymers and other suitable polymers. The backing layer serves the purposes of preventing passage of the drug and/or environmental moisture through the outer surface of the patch, and also for providing any needed support for the system. The backing layer can also provide occlusion, thus increasing the rate of delivery of the drug into the skin. The backing layer is impermeable to the passage of aliskiren or inactive ingredients being present in the formulation and can be either flexible or nonflexible. Suitable materials include, without limitation, polyester, polyethylene terephthalate, some type of nylon, polypropylene, metallized polyester films, polyvinylidene chloride and aluminum foil. Any release liner can be made of the same materials as the backing layer. Hydrogels suitable for matrix type and reservoir transdermal devices are materials that swell when placed in excess water. They do not dissolve in water and maintain three-dimensional networks. Hydrogels are usually made of hydrophilic polymer molecules which are crosslinked either by chemical bonds or other cohesion forces such as ionic interaction, hydrogen bonding or hydrophobic interaction. See, e.g., Park K et al. BIODEGRADABLE HYDROGELS FOR DRUG DELIVERY. Technomic Publishing Co., Inc. 1993. Examples of hydrogels are polyvinylpyrrolidone and cellulose hydrogels such as methylcellulose, hydroxyethylcellulose, hydroxyethylmethylcellulose, carboxymethylcellulose, ethylcellulose, hydroxypropylmethylcellulose, hydroxypropylcellulose and microcrystalline cellulose (colloidal), also included are guar gum, gum arabic, agar, tragacanth, carrageenan, xanthan gum, algin, carbomer, dextran and chitin. It may be additionally therapeutically useful to include at least one transdermal permeation-enhancing substance(s) in order to increase the amount of aliskiren which may permeate the skin and reach the systemic circulation, or in order to reduce the size of the patch. In exemplary fashion, without limitation, such enhancing substances might include alcohols, such as short chain alcohols, e.g. ethanol and the like, long chain fatty alcohols, e.g. lauryl alcohols, and the like, and poly-alcohols, e.g. propylene glycol, glycerin and the like; amides; amino acids; essential oils, fatty acids and fatty acid esters; macrocyclic compounds; phospholipid and phosphate compounds, sulfoxides; and fatty acid ethers. For a useful overview of enhancers, see e.g., Santus G C et al. Transdermal enhancer patent literature. J CONTROL RELEASE 1993; 25: 1-20, and Smith E W et al. PERCUTANEOUS PENETRATION ENHANCERS. CRC Press Inc. 1995.

The invention relates to the use of transdermally administered compounds in the treatment of disorders responsive to the inhibition of renin, but especially hypertension. The compounds transdermally administered in the present invention can be further used in the treatment of congestive heart failure, cardiac hypertrophy, cardiac fibrosis, cardiomyopathy post-infarction, complications resulting from diabetes, such as nephropathy, vasculopathy and neuropathy, diseases of the coronary vessels, restenosis following angioplasty, raised intraocular pressure, glaucoma, abnormal vascular growth, hyperaldosteronism, anxiety states and cognitive disorders.

The doses to be administered are from approximately ≦2 mg to approximately 600 mg, per person per day, preferably as a single dose. Usually, children receive about half of the 20 adult dose. The dose necessary for each individual can be monitored, for example by measuring the serum/plasma concentration of the active ingredient, and adjusted to an optimum level.

Based on the pharmacokinetic properties of aliskiren in the population to be treated, the clinical efficacy profile, the age and body weight range to be covered (e.g., pediatric patients) and the properties of the patch formulation required, patch areas are mainly expected to be in the range 1-10 cm2, preferably about 4 cm2. Further, when aliskiren is administered in a transdermal device, the device should preferably be occlusive. Various carriers and vehicles for aliskiren may be used in the transdermal administration. One such carrier is cyclodextrin, more particularly, β-cyclodextrin.

The following examples are provided to illustrate the present invention without limiting the same hereto.

### Example 1

### Animal Studies Comparing the Bioavailability of Aliskiren after Oral and Transdermal Administration of Aliskiren in Sprague-Dawley Rats

Male Sprague-Dawley rats (300-400 g body weight) were purchased from Charles River Canada Corporation (188 Rue LaSalle, St-Constant, QC, J5A 1Y2, Canada). All animals were maintained under identical conditions and had free access to standard pelleted rat chow and water. For oral dosing, aliskiren was dissolved in 0.5% methocel and administered via feeding tubes. The compound was dosed in a single bolus of 3 mg/5 ml/kg or 25 mg/5 ml/kg. For intravenous (IV) 5 dosing, aliskiren was dissolved in 60% PEG 200 and administered in a single bolus at 0.5 mg/1 ml/kg. For transdermal delivery, aliskiren was dissolved in 100% DMSO, and applied (single application of 250 μl of solution) onto the shaved skin of the rat. The rat was lightly sedated under 2.5% isoflurane anesthesia, and its back was shaved over a 4 cm² area. The animal was returned to its cage to recover from anesthesia. Twenty-four hours later, the rat was lightly sedated under 2.5% isoflurane anesthesia, and the shaved area was disinfected with three passes of ethanol. After evaporation of the ethanol, a volume of 254.1 of 100% DMSO only, or of the compound dissolved in a 100% DMSO solution was applied over the shaved area using a micropipette. After complete evaporation of the DMSO solution (within 5 min after application), an occlusive transparent, waterproof film (OpSite) was taped to the back of the animal over the shaved area, and a jacket was fitted on the animal. Isoflurane inhalation was stopped, and the animal individually caged. The jacket was removed four hours after application of the compound solution.

A blood sample (0.4 ml) was taken by tail or jugular vein bleed for the determination of compound levels, 1 h, 2 h, 4 h, 6 h, 8 h, 10 h and 24 h after either oral gavage or application on the skin of the compound dissolved in 100% DMSO solution. The animal was euthanized after the 24 h time point. The 100% DMSO application was very well tolerated, and no cutaneous or subcutaneous lesion was observed at necropsy. The plasma was separated by centrifugation and stored at −20° C. pending analysis. Plasma samples were analyzed for aliskiren concentrations using liquid chromatography coupled to mass spectrometry (LC-MS/MS). Results are expressed as the average of the values obtained in four animals per group and shown below in Table 1. Transdermal delivery of aliskiren increased its bioavailability (F) by 4 to 54 fold (depending on the dose used), when compared to oral delivery.

TABLE 1
Comparison of aliskiren bioavailability after PO vs TD delivery in
male Sprague-Dawley (SD) rats.

| | | Dose | AUC | |
|---|---|---|---|---|
| Species | Route | (mg/kg) | (μM * hr) | F (%) |
| male SD | PO | 3 | 0.0021 | 0.1 |
| | TD | 2.9 | 0.084 | 5.4 |
| | PO | 25 | 0.058 | 0.43 |
| | TD | 29 | 0.25 | 1.6 |

### Example 2

### Exemplary Patch Formulation

A transdermal dosage form may be prepared as follows. Aliskiren is added to a suitable solvent and mixed until dissolved. To this solution, a copolymer (e.g., acrylate) is added and the substances are mixed until a uniform coating formulation results. The coating formulation is then coated onto a liner (e.g. silicone). The liner is oven dried and then laminated onto a laminate film of polyethylene terephthalate and ethylene vinyl acetate (e.g., a product such as Scotchpak9732, 3M, St. Paul, Minn.).

Alternatively Phase I formulations can be simple solutions in acceptable dermal vehicles e.g. propylene glycol, with or without permeation enhancers e.g., oleic acid. These formulations can be applied on to skin with an applicator and covered with occluding patch or bandage. Such simple formulations can afford a quick read of clinical proof of concept.

### Example 3

### Animal Studies with Double Transgenic Rats

Female double transgenic (dtg) rats, which are transgenic for human renin and angiotensin (see, e.g., Bohlender et al., J AM SOC NEPHROL 11:2056 (2000)) were used. All animals were maintained under identical conditions and had free access to normal pelleted rat chow and water. Rats were initially treated with enalapril (1 mg/kg/day), starting 3 weeks after birth and continuing for 2 months. After approximately two weeks following cessation of enalapril treatment, the double transgenic rats are hypertensive, with mean arterial blood pressures in the range of 160-170 mmHg.

Transmitter implantation—The rats were anesthetized using isoflurane (via inhalation, 2-3%) The pressure transmitter was implanted under aseptic conditions into the peritoneal cavity with the sensing catheter placed in the descending aorta below the renal arteries pointing upstream. The transmitter was sutured to the abdominal musculature, the skin closed, and the rats were individually housed in a cage, placed on a telemetry receiver pad to enable collection of the blood pressure data during recovery from anesthesia and thereafter. The rats were singly caged for the duration of the recording of telemetry data.

Telemetry-System—Telemetry units were obtained from Data Sciences (St. Paul, Minn.). The implanted sensor consisted of a fluid-filled catheter (0.7 mm diameter, 8 cm long) connected to a highly stable low-conductance strain-gauge pressure transducer, which measured the absolute arterial pressure relative to a vacuum, and a radio-frequency transmitter. The tip of the catheter was filled with a viscous gel that prevents blood reflux and was coated with an antithrombogenic film. The implants (length=2.5 cm, diameter=1.2 cm) weighed 9 g and have a typical battery life of 6 months. A receiver platform (model RPC-1 from Data Sciences) connected the radio signal to digitized input that was sent to a dedicated personal computer. Arterial pressures were calibrated by using an input from an ambient-pressure reference (APR-1, Data Sciences). Systolic, mean, and diastolic blood pressures were expressed in millimeter of mercury (mmHg).

Drug administration—For oral dosing, aliskiren was dissolved in 0.5% methocel and administered via feeding tubes. The compound was dosed in a single bolus of 3 mg/5 ml/kg or 30 mg/5 ml/kg. After dosing, the rat was returned to the cage. Blood pressure data were collected up to 7 days after oral dosing.

For transdermal delivery, aliskiren was dissolved in 100% DMSO to be applied in a single application of 250 μl of solution. The rat was lightly sedated under 2.5% isoflurane anesthesia, and its back was shaved over a 4 cm² area. The animal was returned to the cage to recover from anesthesia. Twenty-four hours later, the rat was lightly sedated under 2.5% isoflurane anesthesia, and the shaved area disinfected with 3 passes of ethanol.

After evaporation of the ethanol, a volume of 250 μl of 100% DMSO only, or of the compound dissolved in a 100% DMSO solution was applied over the shaved area using a micropipette. After complete evaporation of the DMSO solution (within 5 min after application), an occlusive transparent, waterproof film (OpSite) was taped to the back of the animal over the shaved area, and a jacket was fitted on the animal. Isoflurane inhalation was stopped, and the animal individually caged. Blood pressure data was collected up to 5 days after application of the compound/DMSO solution.

Pharmacokinetics and biomarkers—A blood sample (0.3 ml) was taken by tail bleed or jugular intravenous 4 h and 24 h after TD delivery to determine compound levels and plasma renin activity (PRA).

Hemodynamic measurements—For oral delivery, double transgenic rats with implanted pressure transmitters were dosed by oral gavage with a single bolus of vehicle (5 ml/kg) or of the test substance (30 mg/5 ml/kg) (n=6 per group).

For transdermal delivery, double transgenic rats with implanted pressure transmitters were dosed with a single application of vehicle (250 μl of 100% DMSO; n=4) or of the test substance (10 mg in 250 μl of 100% DMSO, i.e. 36 mg/kg; n=5).

The mean arterial blood pressure was continuously monitored. The effect of the test substance is expressed as maximal decrease of mean arterial pressure (MAP) in the treated group versus the control group.

Bioavailability—Table 2 summarizes the bioavailability (estimated as area under the curve, or AUC) of the active drug in systemic circulation is shown comparing oral and transdermal delivery. Transdermal delivery of aliskiren increased the AUC by 70 fold at the dose used.

TABLE 2
AUCs for aliskiren after PO or TD delivery in telemetrized
female dTG rats

| | | Dose | AUC_{24 h} |
|---|---|---|---|
| Species | Route | (mg/kg) | (μM * hr) |
| female | PO | 30 | <0.021 |
| dTG | TD | 36 | 1.5 |

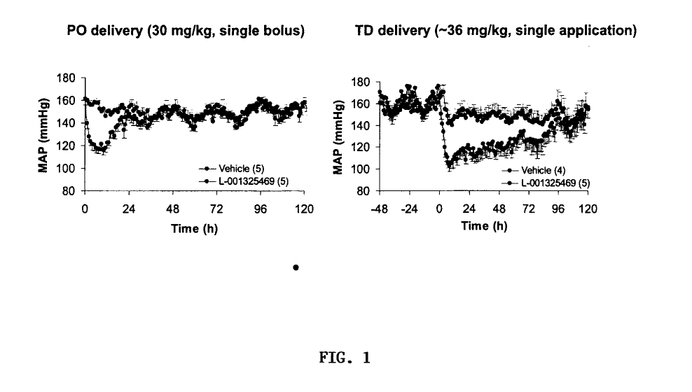

Mean Arterial Pressure—The effects of aliskiren on mean arterial pressure (MAP) were measured with a telemetry system in nonrestrained conscious rats as described above. During the recording period, the animals were kept in a separate room to avoid ambient stress. Exemplary results are shown in FIG. 1. The maximal MAP decrease was comparable between PO and TD delivery, but TD delivery provided a more sustained (>3 days) MAP reduction. (Table 3).

TABLE 3
Comparison of the efficacy of TD vs. PO delivery of aliskiren

| | ABC_{36 h} | max MAP | duration | PRA inhibition |
|---|---|---|---|---|
| Route | (mmHg * hr) | decrease (mmHg) | (days) | at 4 h (%) |
| PO | 630 | 40 | 1.5 | n.a. |
| TD | 1213 | 40 | >3 | 97 |

What is claimed is:

1. A dosage form for transdermal administration, comprising at least one compound for treating hypertension, which is selected from the group consisting of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide and a stereoisomer thereof, or mixture thereof, salts thereof, prodrugs thereof, and metabolites thereof, and a transdermal administration device selected from the group consisting of a reservoir, a matrix, a drug-in-adhesive, a multi-laminate, a polymer-system with no foils, a iontophoretic device, and combinations thereof, an electroporation, a sonophoration, an electroosmosis, an electroincorporation, microneedle and a jet injection device.

2. The dosage form according to claim 1, wherein said compound for treating hypertension is present in a complex with cyclodextrin.

3. The dosage form according to claim 1, further comprising a substance enhancing transdermal penetration.

4. The dosage form according to claim 1, further comprising a substance reducing irritant reactions.

5. The dosage form according to claim 1, wherein said device is occlusive.

6. A method for treating hypertension which comprises the step of transdermally administering with a dosage form of claim 1 at least one compound selected from the group consisting of (R)-aliskiren or the racemate thereof, salts thereof, prodrugs thereof, and metabolites thereof.

7. The dosage form according to claim 1, further comprising at least one pharmaceutically acceptable carrier.

8. The dosage form according to claim 1, wherein the transdermal administration device is a combination of a drug-in-adhesive device and a reservoir device.

9. The method according to claim 6, further comprising at least one pharmaceutically acceptable carrier.
